(19) United States
(12) Patent Application Publication
(10) Pub. No.: US 2017/0252228 A1
(43) Pub. Date: Sep. 7, 2017

(54) STRETCHABLE LAMINATE, AND ARTICLE INCLUDING SAME

(71) Applicant: NITTO DENKO CORPORATION, Ibaraki-shi, Osaka (JP)

(72) Inventors: Shinsuke IKISHIMA, Ibaraki-shi (JP); Shou UCHIDA, Ibaraki-shi (JP); Hiroyuki KONDOU, Ibaraki-shi (JP)

(73) Assignee: NITTO DENKO CORPORATION, Ibaraki-shi, Osaka (JP)

(21) Appl. No.: 15/506,607

(22) PCT Filed: Aug. 18, 2015
(86) PCT No.: PCT/JP2015/073102

§ 371 Date: Feb. 24, 2017

(30) Foreign Application Priority Data
Aug. 26, 2014 (JP) 2014-171313

(51) Int. Cl. A61F 13/49; B32B 5/02; B32B 27/08; B32B 27/12; B32B 27/32; A41D 13/11; B32B 7/12

(52) U.S. Cl. CPC A61F 13/49009; A41D 13/11; B32B 5/022; B32B 7/12; B32B 27/12; B32B 27/327; B32B 27/08; B32B 2274/00; B32B 2307/51; B32B 2307/718; B32B 2555/02; B32B 2437/04

(57) ABSTRACT

Provided is a stretchable laminate excellent in oil resistance. Also provided is an article including such stretchable laminate. The stretchable laminate of the present invention includes: an elastomer layer; and a non-woven fabric layer arranged on at least one side of the elastomer layer, in which: the elastomer layer includes a plurality of layers; and at least one outer layer of the plurality of layers contains an olefin-based elastomer.

### TECHNICAL FIELD

The present invention relates to a stretchable laminate and an article including the stretchable laminate.

### BACKGROUND ART

Various stretchable laminates are proposed for articles such as sanitary articles, for example, diapers and masks (see, for example, Patent Literatures 1 and 2).

As such materials, a stretchable laminate including an elastomer layer and a non-woven fabric layer arranged on at least one side of the elastomer layer has been proposed. In such stretchable laminate, the elastomer layer and the non-woven fabric layer are generally bonded onto each other with an adhesive or a pressure-sensitive adhesive.

However, the related-art stretchable laminate including the elastomer layer and the non-woven fabric layer arranged on at least one side of the elastomer layer involves a problem in that its oil resistance is low. For example, the stretchable laminate involves a problem in that the elastomer layer in the stretchable laminate is perforated with a hole or broken by an oil component in an adhesive or a pressure-sensitive adhesive or by an aliphatic oil, such as a baby oil.

### CITATION LIST

### Patent Literature

[PTL 1] JP 2012-187857 A

[PTL 2] JP 3830818 B2

### SUMMARY OF INVENTION

### Technical Problem

The present invention has been made to solve the problem of the related art, and an object of the present invention is to provide a stretchable laminate excellent in oil resistance. Another object of the present invention is to provide an article including such stretchable laminate.

### Solution to Problem

A stretchable laminate according to one embodiment of the present invention includes: an elastomer layer; and a non-woven fabric layer arranged on at least one side of the elastomer layer, in which: the elastomer layer includes a plurality of layers; and at least one outer layer of the plurality of layers contains an olefin-based elastomer.

In a preferred embodiment, the elastomer layer includes three layers, and outer layers on both sides of the elastomer layer each contain the olefin-based elastomer.

In a preferred embodiment, a content of the olefin-based elastomer in the outer layer is from 50 wt % to 100 wt %.

In a preferred embodiment, the content of the olefin-based elastomer in the outer layer is from 80 wt % to 100 wt %.

In a preferred embodiment, the content of the olefin-based elastomer in the outer layer is from 95 wt % to 100 wt %.

In a preferred embodiment, the olefin-based elastomer includes an α-olefin-based elastomer.

In a preferred embodiment, the α-olefin-based elastomer includes at least one kind selected from an ethylene-based elastomer, a propylene-based elastomer, and a 1-butene-based elastomer.

In a preferred embodiment, the α-olefin-based elastomer includes a propylene-based elastomer.

In a preferred embodiment, the α-olefin-based elastomer is produced by using a metallocene catalyst.

In a preferred embodiment, an elastomer layer except the outer layer containing the olefin-based elastomer in the elastomer layer contains 50 wt % to 100 wt % of an elastomer resin.

In a preferred embodiment, the elastomer layer except the outer layer containing the olefin-based elastomer in the elastomer layer contains 80 wt % to 100 wt % of an elastomer resin.

In a preferred embodiment, the elastomer layer except the outer layer containing the olefin-based elastomer in the elastomer layer contains 95 wt % to 100 wt % of an elastomer resin.

In a preferred embodiment, the elastomer layer except the outer layer containing the olefin-based elastomer in the elastomer layer contains an elastomer resin, and the elastomer resin includes at least one kind selected from an olefin-based elastomer, a styrene-based elastomer, a vinyl chloride-based elastomer, a urethane-based elastomer, an ester-based elastomer, and an amide-based elastomer.

In a preferred embodiment, the elastomer resin includes at least one kind selected from the olefin-based elastomer and the styrene-based elastomer.

In a preferred embodiment, the elastomer layer has a thickness of from 20 μm to 200 μm.

In a preferred embodiment, the elastomer layer has a thickness of from 30 μm to 100 μm.

In a preferred embodiment, the non-woven fabric layer is formed of a non-woven fabric having a basis weight of from 10 gsm to 30 gsm.

In a preferred embodiment, the stretchable laminate further includes a hot-melt pressure-sensitive adhesive between the elastomer layer and the non-woven fabric layer.

A stretchable laminate according to one embodiment of the present invention includes: an elastomer layer; and a non-woven fabric layer arranged on at least one side of the elastomer layer, in which when the stretchable laminate is bonded and fixed onto a glass plate in a state of being extended by 100%, and 0.5 mL of a baby oil (manufactured by Pigeon Corporation, Baby Oil P, main component: caprylic/capric triglyceride) is dropped onto a surface of the stretchable laminate, the stretchable laminate is free from rupturing 10 minutes after the dropping.

An article according to one embodiment of the present invention includes the stretchable laminate according to the embodiment of the present invention.

### BRIEF DESCRIPTION OF DRAWINGS

FIG. 1 is a schematic sectional view of a stretchable laminate according to a preferred embodiment of the present invention.

FIG. 2 is a schematic view for illustrating another stretchable laminate according to a preferred embodiment of the present invention.

FIG. 3 is a schematic sectional view for illustrating one preferred embodiment of an elastomer layer.

FIG. 4 is a schematic view of a state in which a non-woven fabric is coated with a hot-melt pressure-sensitive adhesive in a striped manner in a flow direction of a production line as viewed from a top surface thereof.

### DESCRIPTION OF EMBODIMENTS

### <<<<Stretchable Laminate>>>>

A stretchable laminate of the present invention is a stretchable laminate including an elastomer layer and a non-woven fabric layer arranged on at least one side of the elastomer layer. The stretchable laminate of the present invention may include any appropriate other layer as long as the stretchable laminate includes an elastomer layer and a non-woven fabric layer arranged on at least one side of the elastomer layer and the effects of the present invention are not impaired. The number of such any appropriate other layers may be only one, or may be two or more.

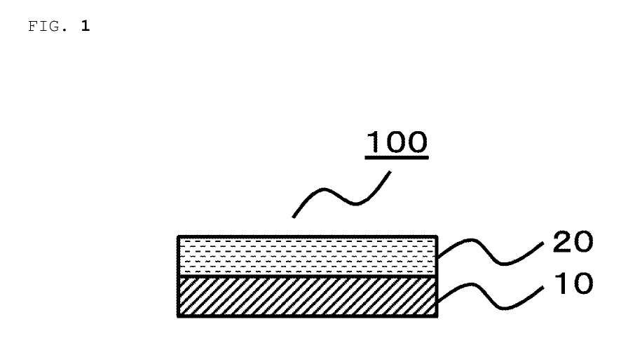

FIG. 1 is a schematic sectional view of a stretchable laminate according to a preferred embodiment of the present invention. A stretchable laminate 100 illustrated in FIG. 1 includes an elastomer layer 10 and a non-woven fabric layer 20 arranged on only one side of the elastomer layer 10. A material for bonding the elastomer layer 10 and the non-woven fabric layer 20 may be present therebetween. Examples of such material include an adhesive, a pressure-sensitive adhesive, and a hot-melt pressure-sensitive adhesive.

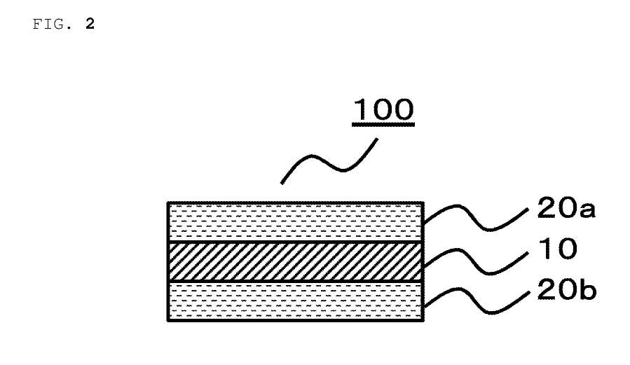

FIG. 2 is a schematic sectional view of another stretchable laminate according to a preferred embodiment of the present invention. A stretchable laminate 100 illustrated in FIG. 2 includes an elastomer layer 10, a non-woven fabric layer 20a arranged on one side of the elastomer layer 10, and a non-woven fabric layer 20b arranged on the elastomer layer 10 on an opposite side to the non-woven fabric layer 20a. A material for bonding the elastomer layer 10 and the non-woven fabric layer 20a and/or for bonding the elastomer layer 10 and the non-woven fabric layer 20b may be present therebetween. Examples of such material include an adhesive, a pressure-sensitive adhesive, and a hot-melt pressure-sensitive adhesive.

The thickness of the stretchable laminate of the present invention varies depending on the thickness of the elastomer layer or the thickness of the non-woven fabric layer and is preferably from 1.0 mm to 0.1 mm, more preferably from 0.8 mm to 0.15 mm, still more preferably from 0.6 mm to 0.15 mm, particularly preferably from 0.5 mm to 0.2 mm, most preferably from 0.45 mm to 0.2 mm. When the thickness of the stretchable laminate of the present invention falls within such range, the laminate can be easily used as a material used in articles such as sanitary articles, for example, diapers and masks.

### <<Elastomer Layer>>

The elastomer layer includes a plurality of layers. Any appropriate number may be adopted as the number of such plurality of layers. The number of such plurality of layers is preferably from 2 to 5, more preferably from 2 to 4, still more preferably from 2 or 3, particularly preferably 3.

In the elastomer layer, at least one outer layer of the plurality of layers contains an olefin-based elastomer. That is, a mode in which only one outer layer of the plurality of layers contains the olefin-based elastomer is permitted, and a mode in which both outer layers of the plurality of layers each contain the olefin-based elastomer is also permitted. When, in the elastomer layer including the plurality of layers, at least one outer layer of the plurality of layers contains the olefin-based elastomer, a stretchable laminate excellent in oil resistance can be provided.

The olefin-based elastomer may be only one kind of elastomer, or may be a blend of two or more kinds of elastomers.

In addition, when, in the elastomer layer including the plurality of layers, at least one outer layer of the plurality of layers contains the olefin-based elastomer, heat stability is improved and hence, for example, heat decomposition at the time of film formation in producing the stretchable laminate of the present invention can be suppressed. In addition, when, in the elastomer layer including the plurality of layers, at least one outer layer of the plurality of layers contains the olefin-based elastomer, storage stability is improved and hence the fluctuation of values for physical properties during the storage of the stretchable laminate of the present invention can be suppressed.

In addition, when, in the elastomer layer including the plurality of layers, the olefin-based elastomer is adopted for at least one outer layer of the plurality of layers, steps in the production of the elastomer layer can be simplified, and hence processing cost can be reduced. This is because of the following reason: when the olefin-based elastomer is adopted, extrusion molding can be performed by using fewer kinds of resins in the production of the elastomer layer, and hence the need for the production of a master batch can be eliminated.

The content of the olefin-based elastomer in the outer layer containing the olefin-based elastomer is preferably from 50 wt % to 100 wt %, more preferably from 70 wt % to 100 wt %, still more preferably from 80 wt % to 100 wt %, particularly preferably from 90 wt % to 100 wt o, most preferably from 95 wt % to 100 wt % in terms of the expression of the effects of the present invention. When the content of the olefin-based elastomer in the outer layer containing the olefin-based elastomer falls within the range, a stretchable laminate more excellent in oil resistance can be provided.

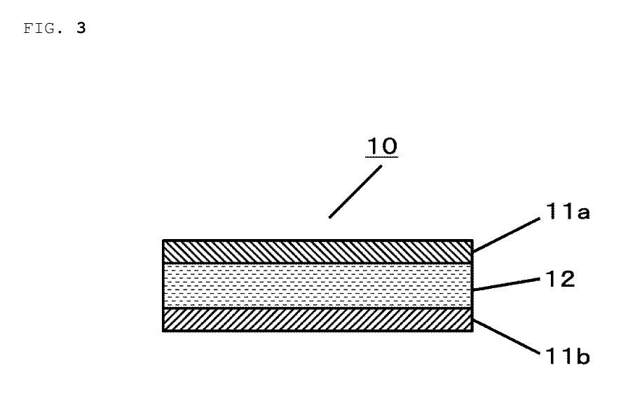

FIG. 3 is a schematic sectional view for illustrating one preferred embodiment of the elastomer layer. In FIG. 3, the elastomer layer 10 is formed of three layers. Outer layers 11a and 11b on both sides of the elastomer layer each contain an olefin-based elastomer, and the elastomer layer includes an intermediate layer 12 between the outer layer 11a and the outer layer 11b.

Examples of the olefin-based elastomer in the outer layer include an olefin block copolymer, an olefin random copolymer, an ethylene copolymer, a propylene copolymer, an ethylene olefin block copolymer, a propylene olefin block copolymer, an ethylene olefin random copolymer, a propylene olefin random copolymer, an ethylene propylene random copolymer, an ethylene (1-butene) random copolymer, an ethylene (1-pentene) olefin block copolymer, an ethylene (1-hexene) random copolymer, an ethylene (1-heptene) olefin block copolymer, an ethylene (1-octene) olefin block copolymer, an ethylene (1-nonene) olefin block copolymer, an ethylene (1-decene) olefin block copolymer, a propylene ethylene olefin block copolymer, an ethylene (α-olefin) copolymer, an ethylene (α-olefin) random copolymer, an ethylene (α-olefin) block copolymer, and combinations thereof.

The olefin-based elastomer in the outer layer has a density of preferably from 0.890 g/cm³ to 0.830 g/cm³, more preferably from 0.888 g/cm³ to 0.835 g/cm³, still more preferably from 0.886 g/cm³ to 0.835 g/cm³, particularly preferably from 0.885 g/cm³ to 0.840 g/cm³, most preferably from 0.885 g/cm³ to 0.845 g/cm³. When the olefin-based elastomer whose density falls within the range described above is adopted for the outer layer, a stretchable laminate more excellent in oil resistance can be provided, and the heat stability is further improved, and hence, for example, the heat decomposition at the time of film formation in producing the stretchable laminate of the present invention can be further suppressed. In addition, the storage stability is further improved, and hence the fluctuation of values for physical properties during the storage of the stretchable laminate of the present invention can be further suppressed. Further, the steps in the production of the elastomer layer can be further simplified, and hence the processing cost can be further reduced.

The olefin-based elastomer in the outer layer has a MFR at 230° C. and 2.16 kgf of preferably from 1.0 g/10 min to 25.0 g/10 min, more preferably from 2.0 g/10 min to 23.0 g/10 min, still more preferably from 2.0 g/10 min to 21.0 g/10 min, particularlypreferably from 2.0 g/10 min to 20.0 g/10 min, most preferably from 2.0 g/10 min to 19.0 g/10 min. When the olefin-based elastomer whose MFR falls within the range described above is adopted for the outer layer, a stretchable laminate having more excellent in oil resistance can be provided, and the heat stability is further improved, and hence, for example, the heat decomposition at the time of film formation in producing the stretchable laminate of the present invention can be further suppressed. In addition, the storage stability is further improved, and hence the fluctuation of values for physical properties during the storage of the stretchable laminate of the present invention can be further suppressed. Further, the steps in the production of the elastomer layer can be further simplified, and hence the processing cost can be further reduced.

The olefin-based elastomer in the outer layer is specifically preferably an α-olefin-based elastomer. That is, the α-olefin-based elastomer is a copolymer of two or more kinds of α-olefins and has elastomer characteristics. Of such α-olefin-based elastomers, any one selected from an ethylene-based elastomer, a propylene-based elastomer, and a 1-butene-based elastomer is more preferred. When such α-olefin-based elastomer is adopted as the olefin-based elastomer, a stretchable laminate more excellent in oil resistance can be provided, and the heat stability is further improved, and hence, for example, the heat decomposition at the time of film formation in producing the stretchable laminate of the present invention can be further suppressed. In addition, the storage stability is further improved, and hence the fluctuation of values for physical properties during the storage of the stretchable laminate of the present invention can be further suppressed. Further, the steps in the production of the elastomer layer can be further simplified, and hence the processing cost can be further reduced.

Of the α-olefin-based elastomers each serving as the olefin-based elastomer in the outer layer, a propylene-based elastomer is particularly preferred. When the propylene-based elastomer is adopted as the olefin-based elastomer, a stretchable laminate more excellent in oil resistance can be provided, and the heat stability is further improved, and hence, for example, the heat decomposition at the time of film formation in producing the stretchable laminate of the present invention can be further suppressed. In addition, the storage stability is further improved, and hence the fluctuation of values for physical properties during the storage of the stretchable laminate of the present invention can be further suppressed. Further, the steps in the production of the elastomer layer can be further simplified, and hence the processing cost can be further reduced.

Such α-olefin-based elastomer as described above is also available as a commercial product. Examples of such commercial product include some products in the “Tafmer” (trademark) series (such as Tafmer PN-3560) manufactured by Mitsui Chemicals, Inc., and some products in the “Vistamaxx” (trademark) series (such as Vistamaxx 6202 and Vistamaxx 7010) manufactured by Exxon Mobil Corporation.

The α-olefin-based elastomer serving as the olefin-based elastomer in the outer layer is preferably produced by using a metallocene catalyst. With the α-olefin-based elastomer produced by using a metallocene catalyst, a stretchable laminate more excellent in oil resistance can be provided, and the heat stability is further improved, and hence, for example, the heat decomposition at the time of film formation in producing the stretchable laminate of the present invention can be further suppressed. In addition, the storage stability is further improved, and hence the fluctuation of values for physical properties during the storage of the stretchable laminate of the present invention can be further suppressed. Further, the steps in the production of the elastomer layer can be further simplified, and hence the processing cost can be further reduced.

Any appropriate elastomer layer may be adopted as an elastomer layer except the outer layer containing the olefin-based elastomer in the elastomer layer including the plurality of layers (e.g., the intermediate layer 12 of FIG. 3) as long as the effects of the present invention are not impaired. As an elastomer resin serving as a main component of such elastomer layer, there are given, for example, an olefin-based elastomer, a styrene-based elastomer, a vinyl chloride-based elastomer, a urethane-based elastomer, an ester-based elastomer, and an amide-based elastomer.

The content of the elastomer resin serving as a main component in the elastomer layer except the outer layer containing the olefin-based elastomer in the elastomer layer including the plurality of layers is preferably from 50 wt % to 100 wt %, more preferably from 70 wt % to 100 wt %, still more preferably from 80 wt % to 100 wt %, particularly preferably from 90 wt % to 100 wt %, most preferably from 95 wt % to 100 wt %. When the content of the elastomer resin serving as a main component in the elastomer layer except the outer layer containing the olefin-based elastomer in the elastomer layer including the plurality of layers falls within the range, the elastomer layer can express a sufficient elastomer characteristic.

The number of the elastomer layers except the outer layer containing the olefin-based elastomer in the elastomer layer including the plurality of layers may be one, or may be two or more.

The elastomer layer may contain any appropriate other component as long as the effects of the present invention are not impaired. Examples of such other component include any other polymer, a tackifier, a plasticizer, an antidegradant, a pigment, a dye, an antioxidant, an antistatic agent, a lubricant, a blowing agent, a heat stabilizer, a light stabilizer, an inorganic filler, and an organic filler. The number of kinds of those components may be only one, or may be two or more. The content of the other component in the elastomer layer is preferably 10 wt % or less, more preferably 7 wt % or less, still more preferably 5 wt % or less, particularly preferably 2 wt % or less, most preferably 1 wt % or less.

The thickness of the elastomer layer is preferably from 200 μm to 20 μm, more preferably from 160 μm to 30 μm, still more preferably from 140 μm to 30 μm, particularly preferably from 120 μm to 30 μm, most preferably from 100 μm to 30 μm. When the thickness of the elastomer layer falls within such range, a stretchable laminate having more excellent fittability can be provided.

### <<Non-woven Fabric Layer>>

Any appropriate non-woven fabric layer may be adopted as the non-woven fabric layer as long as the effects of the present invention are not impaired. The number of kinds of non-woven fabrics constituting the non-woven fabric layer may be only one, or may be two or more.

Examples of the non-woven fabric constituting the non-woven fabric layer include a spunbonded non-woven web, a fluffy non-woven fabric (such as a non-woven fabric obtained by a thermal bonding method, a bonding joining method, or a spunlace method), a meltblown non-woven web, a spunlace non-woven web, a spunbonded meltblown spunbonded non-woven web, a spunbonded meltblown meltblown spunbonded non-woven web, an unjoined non-woven web, an electrospun non-woven web, a flashspun non-woven web (such as TYVEK™ from DuPont), and a carded non-woven fabric.

For example, the non-woven fabric constituting the non-woven fabric layer may contain fiber of polypropylene, polyethylene, polyester, polyamide, polyurethane, an elastomer, rayon, cellulose, acrylic, a copolymer thereof, or a blend thereof, or a mixture thereof, or any other polyolefin.

The non-woven fabric constituting the non-woven fabric layer may contain fiber that is a homogeneous structural body, or may contain a bicomponent structural body, such as a sheath/core structure, a side-by-side structure, a sea-island structure, and any other bicomponent structure. Detailed descriptions of the non-woven fabric may be found in, for example, “Nonwoven Fabric Primer and Reference Sampler,” E.A. Vaughn, Association of the Nonwoven Fabrics Industry, third edition (1992).

The basis amount of the non-woven fabric constituting the non-woven fabric layer is preferably 150 gsm or less, more preferably 100 gsm or less, still more preferably 50 gsm or less, particularly preferably from 10 gsm to 30 gsm.

### <<Production of Stretchable Laminate of the Present Invention>>

In the production of the stretchable laminate of the present invention, when the elastomer layer and the non-woven fabric layer are directly laminated (for example, in the cases of FIG. 1 and FIG. 2), the elastomer layer and the non-woven fabric layer need to be bonded onto each other. Examples of such bonding method include: (1) a method involving laminating an elastomer layer formed by extrusion from a T-die of an extruder and a non-woven fabric layer separately fed from a rolled body; (2) a method involving laminating an elastomer layer and a non-woven fabric layer by co-extrusion; (3) a method involving bonding an elastomer layer and a non-woven fabric layer, which are prepared separately, with an adhesive; (4) a method involving forming a non-woven fabric layer on an elastomer layer formed by any appropriate method through use of a meltblown method or the like; and (5) thermally laminating or ultrasonically welding an elastomer layer and a non-woven fabric layer.

The elastomer layer and the non-woven fabric layer may be bonded onto each other with a hot-melt pressure-sensitive adhesive. When the hot-melt pressure-sensitive adhesive is used, the need to add a tackifier as a component of the elastomer layer is reduced, and hence, for example, the extrusion stability is improved, a problem in that the tackifier adheres to a forming roll can be suppressed, and a contamination problem of the production line by volatile matter contamination or the like caused by the tackifier can be suppressed.

When the hot-melt pressure-sensitive adhesive is used for bonding the elastomer layer and the non-woven fabric layer, in the case of applying the method (1) described above, the non-woven fabric layer separately fed from a rolled body may be coated with the hot-melt pressure-sensitive adhesive before being laminated with the elastomer layer.

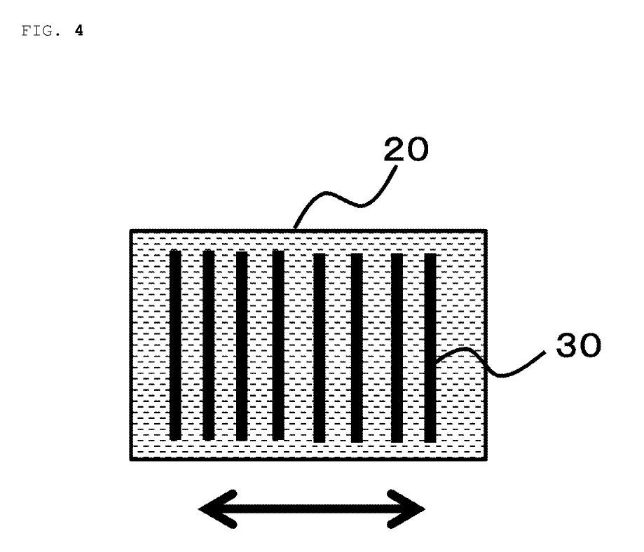

When the hot-melt pressure-sensitive adhesive is used for bonding the elastomer layer and the non-woven fabric layer, it is not necessary to coat the whole surface of the non-woven fabric layer with the hot-melt pressure-sensitive adhesive. For example, as illustrated in FIG. 4, the non-woven fabric layer 20 may be coated with a hot-melt pressure-sensitive adhesive 30 in a striped manner in a flow direction of the production line, or the hot-melt pressure-sensitive adhesive 30 may be applied onto the non-woven fabric layer 20 in a dotted manner. Through the coating with the hot-melt pressure-sensitive adhesive in a striped manner, portions in which the hot-melt pressure-sensitive adhesive is present and portions in which the hot-melt pressure-sensitive adhesive is absent are formed in a striped manner, and therefore, particularly in a direction perpendicular to the stripe pattern (in the direction of the arrow of FIG. 3), the stretchability of the stretchable laminate can be further improved.

The stretchable laminate of the present invention may be subjected to treatments referred to as pre-stretching treatment and activation treatment after laminating the elastomer layer and the non-woven fabric layer. Specifically, stretching treatment is performed in a width direction of the stretchable laminate or treatment in which a fiber structure of a part of the region of the non-woven fabric layer is mechanically broken may be performed. When such treatments are performed, the stretchable laminate can be stretched by a smaller force.

### <<Application of Stretchable Laminate of the Present Invention>>

The stretchable laminate of the present invention can be used in any appropriate article in which the effects of the present invention can be effectively utilized. That is, the article of the present invention includes the stretchable laminate of the present invention. Atypical example of such article is a sanitary article. Examples of such sanitary article include a diaper (in particular, an ear portion of a disposable diaper), a supporter, and a mask.

### EXAMPLES

The present invention is hereinafter specifically described by way of Examples. However, the present invention is by no means limited to these Examples. Test and evaluation methods in Examples and the like are as described below. In addition, “part(s)” means “part(s) by weight” and “%” means “wt %” unless otherwise stated.

### <Evaluation of Dropping Oil Resistance>

Each of stretchable laminates obtained in Examples and Comparative Examples was bonded and fixed onto a glass plate in a state of being extended by 100%. 0.5 mL of a baby oil (manufactured by Pigeon Corporation, Baby Oil P, main component: caprylic/capric triglyceride) was dropped onto the surface of the extended stretchable laminate. A stretchable laminate that ruptured 10 minutes after the dropping was evaluated as x, and a stretchable laminate that did not rupture 10 minutes after the dropping was evaluated as ∘.

<40° C. Holding force Test>

667 parts of a propylene/1-butene copolymer (manufactured by REXtac, LLC, trade name: REXTAC RT 2788), 520 parts of a tackifier (manufactured by Kolon Industries, Inc., trade name: SUKOREZ SU-100 S), 100 parts of liquid paraffin (manufactured by Petro yag, trade name: White Oil Pharma Oyster 259), and 13 parts of an antioxidant (manufactured by BASF, trade name: Irganox 1010) were blended to provide a hot-melt pressure-sensitive adhesive. A sheet obtained by applying the hot-melt pressure-sensitive adhesive onto an OPP film (35 μm) at 15 g/m² was cut into a size of 25 mm in width. The resultant sheet was bonded onto each of the non-woven fabric surfaces (one surface side, whole surface applied portion) of the stretchable laminates produced in Examples and Comparative Examples with a width of 25 mm and a length of 15 mm and compressively bonded thereonto by two reciprocations under a load of 1 kg. The resultant was left to stand still at room temperature for 10 minutes after the compressive bonding, and then left to stand still for 30 minutes under an environment of 40° C. The resultant stretchable film was set on a holding force testing machine, and a load of 1 kg was applied to the OPP film side thereof to stretch the film. 0.1 mL of a baby oil (manufactured by Pigeon Corporation, Baby Oil P, main component: caprylic/capric triglyceride) was applied to the surface of the stretched stretchable film. A stretchable film that ruptured and fell after the application was evaluated as ×, and a time required for the falling to occur was measured. A stretchable film that did not rupture or fall even 3 hours after the application was evaluated as ∘.

### <Elasticity Test>

Each of the stretchable laminates obtained in Examples and Comparative Examples was cut in a CD direction so as to have a width of 30 mm. Only a stripe portion thereof was set on a tension testing machine (manufactured by Shimadzu Corporation: AG-20kNG) at a distance between chucks of 40 mm, and was extended by 100% at a tension speed of 300 mm/min. After having been extended by 100%, the laminate was fixed in an extended state and held at room temperature for 10 minutes. After a lapse of 10 minutes, the laminate was released from the extended state, and the initial distance between the chucks, i.e., 40 mm (A) and the width of the film after the test, i.e., (40+α) mm (B) were measured. After that, a fluctuation ratio was calculated from the expression “[{(B)−(A)}/(A)]×100.” A stretchable laminate whose fluctuation ratio was more than 20% was evaluated as ×, and a stretchable laminate whose fluctuation ratio was less than 20% was evaluated as ∘.

### <Forming Conditions>

In Examples and Comparative Examples, an elastomer layer (hereinafter sometimes referred to as elastic film) was formed by extrusion molding by extruding three layers in two types (A layer/B layer/A layer) through use of a T-die molding machine. The extrusion temperatures were set under the following conditions.

A layer: 200° C.

B layer: 200° C.

Die temperature: 200° C. A non-woven fabric (PP carded type, basis amount=24 gsm) was bonded onto both surfaces of the elastic film extruded from the T-die through use of a roll to provide a stretchable laminate. In this case, a hot-melt pressure-sensitive adhesive was applied onto a bonded side of the non-woven fabric so as to alternately have a portion (A) in which the hot-melt pressure-sensitive adhesive had been applied onto the whole surface (7 g/m²) with a width of 30 mm and a portion (B) in which the hot-melt pressure-sensitive adhesive was applied in a striped manner (pressure-sensitive adhesive width: 1 mm, interval: 1 mm) (15 g/m²) with a width of 41 mm.

### Example 1

100 wt % of an olefin-based resin (manufactured by Exxon Mobil Corporation, trade name: Vistamaxx 6202) was loaded into an A layer in an extrusion machine, and a formulation of 65 wt % of an olefin-based resin (manufactured by Exxon Mobil Corporation, trade name: Vistamaxx 6202), 30 wt % of an olefin-based resin (manufactured by Mitsui Chemicals, Inc., trade name: Tafmer PN-3560), and 5 wt % of a white pigment (titanium oxide manufactured by Dupont, trade name: Ti-Pure R103) was loaded into a B layer in the extrusion machine to extrude an elastic film (1) having the construction of A layer/B layer/A layer=9 μm/42 μm/9 μm in total of 60 μm.

The resultant elastic film (1) and a SIS-based hot-melt pressure-sensitive adhesive (manufactured by Bento Bantçilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (1) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 1.

### Example 2

100 wt % of an olefin-based resin (manufactured by Exxon Mobil Corporation, trade name: Vistamaxx 6202) was loaded into an A layer in an extrusion machine, and a formulation of 65 wt % of an olefin-based resin (manufactured by Exxon Mobil Corporation, trade name: Vistamaxx 6202), 30 wt % of an olefin-based resin (manufactured by Mitsui Chemicals, Inc., trade name: Tafmer PN-3560), and 5 wt % of a white pigment (titanium oxide manufactured by Dupont, trade name: Ti-Pure R103) was loaded into a B layer in the extrusion machine to extrude an elastic film (2) having the construction of A layer/B layer/A layer=6.75 μm/31.5 μm/6.75 μm in total of 45 μm.

The resultant elastic film (2) and a SIS-based hot-melt pressure-sensitive adhesive (manufactured by Bento Bantgilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (2) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 1.

### Example 3

A formulation of 50 wt % of an olefin-based resin (manufactured by Exxon Mobil Corporation, trade name: Vistamaxx 6202) and 50 wt % of an olefin-based resin (manufactured by Mitsui Chemicals, Inc., trade name: Tafmer PN-3 56 0) was loaded into an A layer in an extrusion machine, and a formulation of 45 wt % of an olefin-based resin (manufactured by Exxon Mobil Corporation, trade name: Vistamaxx 3000), 50 wt % of an olefin-based resin (manufactured by Mitsui Chemicals, Inc., trade name: Tafmer PN-3560), and 5 wt % of a white pigment (titanium oxide manufactured by Dupont, trade name: Ti-Pure R103) was loaded into a B layer in the extrusion machine to extrude an elastic film (3) having the construction of A layer/B layer/A layer=6.75 μm/31.5 μm/6.75 μm in total of 45 μm.

The resultant elastic film (3) and a SIS-based hot-melt pressure-sensitive adhesive (manufactured by Bento Bantgilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (3) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 1.

### Example 4

A formulation of 50 wt % of an olefin-based resin (manufactured by Exxon Mobil Corporation, trade name: Vistamaxx 6202) and 50 wt % of an olefin-based resin (manufactured by Mitsui Chemicals, Inc., trade name: Tafmer PN-3560) was loaded into an A layer in an extrusion machine, and a formulation of 25 wt % of an olefin-based resin (manufactured by Exxon Mobil Corporation, trade name: Vistamaxx 3000), 70 wt % of an olefin-based resin (manufactured by Mitsui Chemicals, Inc., trade name: Tafmer PN-3560), and 5 wt % of a white pigment (titanium oxide manufactured by Dupont, trade name: Ti-Pure R103) was loaded into a B layer in the extrusion machine to extrude an elastic film (4) having the construction of A layer/B layer/A layer=6.75 μm/31.5 μm/6.75 μm in total of 45 μm.

The resultant elastic film (4) and a SIS-based hot-melt pressure-sensitive adhesive (manufactured by Bento Bantgilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (4) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 1.

### Example 5

100 wt % of an olefin-based resin (manufactured by Exxon Mobil Corporation, trade name: Vistamaxx 7010) was loaded into an A layer in an extrusion machine, and a formulation of 45 wt % of an olefin-based resin (manufactured by Exxon Mobil Corporation, trade name: Vistamaxx 3000), 50 wt % of an olefin-based resin (manufactured by Mitsui Chemicals, Inc., trade name: Tafmer PN-3560), and 5 wt % of a white pigment (titanium oxide manufactured by Dupont, trade name: Ti-Pure R103) was loaded into a B layer in the extrusion machine to extrude an elastic film (5) having the construction of A layer/B layer/A layer=6.75 μm/31.5 μm/6.75 μm in total of 45 μm.

The resultant elastic film (5) and a SIS-based hot-melt pressure-sensitive adhesive (manufactured by Bento Bantgilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (5) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 1.

### Example 6

100 wt % of an olefin-based resin (manufactured by Exxon Mobil Corporation, trade name: Vistamaxx 7010) was loaded into an A layer in an extrusion machine and a formulation of 25 wt % of an olefin-based resin (manufactured by Exxon Mobil Corporation, trade name: Vistamaxx 3000), 70 wt % of an olefin-based resin (manufactured by Mitsui Chemicals, Inc., trade name: Tafmer PN-3560), and 5 wt % of a white pigment (titanium oxide manufactured by Dupont, trade name: Ti-Pure R103) was loaded into a B layer in the extrusion machine to extrude an elastic film (6) having the construction of A layer/B layer/A layer=6.75 μm/31.5 μm/6.75 μm in total of 45 μm.

The resultant elastic film (6) and a SIS-based hot-melt pressure-sensitive adhesive (manufactured by Bento Bantçilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (6) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 1.

### Example 7

100 wt % of an olefin-based resin (manufactured by Exxon Mobil Corporation, trade name: Vistamaxx 7010) was loaded into an A layer in an extrusion machine, and a formulation of 50 wt % of a SIS-based resin (manufactured by Zeon Corporation, trade name: Quintac 3399), 45 wt % of a SBS-based resin (manufactured by Kraton Polymers, Inc., trade name: Kraton D1191), and 5 wt % of a white pigment (titanium oxide manufactured by Dupont, trade name: Ti-Pure R103) was loaded into a B layer in the extrusion machine to extrude an elastic film (7) having the construction of A layer/B layer/A layer=6.75 μm/31.5 μm/6.75 μm in total of 45 μm.

The resultant elastic film (7) and a SIS-based hot-melt pressure-sensitive adhesive (manufactured by Bento Bantgilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (7) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 1.

### Example 8

100 wt % of an olefin-based resin (manufactured by Exxon Mobil Corporation, trade name: Vistamaxx 7010) was loaded into an A layer in an extrusion machine, and a formulation of 50 wt % of a SIS-based resin (manufactured by Zeon Corporation, trade name: Quintac 3399), 45 wt % of a SBS-based resin (manufactured by Kraton Polymers, Inc., trade name: Kraton D1191), and 5 wt % of a white pigment (titanium oxide manufactured by Dupont, trade name: Ti-Pure R103) was loaded into a B layer in the extrusion machine to extrude an elastic film (8) having the construction of A layer/B layer/A layer=9 μm/42 μm/9 μm in total of 60 μm.

The resultant elastic film (8) and a SIS-based hot-melt pressure-sensitive adhesive (manufactured by Bento Bantgilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (8) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 1.

### Example 9

100 wt % of an olefin-based resin (manufactured by Exxon Mobil Corporation, trade name: Vistamaxx 7010) was loaded into an A layer in an extrusion machine, and a formulation of 95 wt % of a SBS-based resin (manufactured by Kraton Polymers, Inc., trade name: Kraton D1191) and 5 wt % of a white pigment (titanium oxide manufactured by Dupont, trade name: Ti-Pure R103) was loaded into a B layer in the extrusion machine to extrude an elastic film (9) having the construction of A layer/B layer/A layer=6.75 μm/31.5 μm/6.75 μm in total of 45 μm.

The resultant elastic film (9) and a SIS-based hot-melt pressure-sensitive adhesive (manufactured by Bento Bantçilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (9) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 1.

### Example 10

100 wt % of an olefin-based resin (manufactured by Exxon Mobil Corporation, trade name: Vistamaxx 6202) was loaded into an A layer in an extrusion machine, and a formulation of 65 wt % of the olefin-based resin (manufactured by Exxon Mobil Corporation, trade name: Vistamaxx 6202), 30 wt % of a SBS-based resin (manufactured by Kraton Polymers, Inc., trade name: Kraton 1730), and 5 wt % of a white pigment (titanium oxide manufactured by Dupont, trade name: Ti-Pure R103) was loaded into a B layer in the extrusion machine to extrude an elastic film (10) having the construction of A layer/B layer/A layer=6.75 μm/31.5 μm/6.75 μm in total of 45 μm.

The resultant elastic film (10) and a SIS-based hot-melt pressure-sensitive adhesive (manufactured by Bento Bantgilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (10) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 1.

### Comparative Example 1

100 wt % of a SIS-based resin (manufactured by Zeon Corporation, trade name: Quintac 3399) was loaded into an A layer in an extrusion machine, and a formulation of 95 wt % of the SIS-based resin (manufactured by Zeon Corporation, trade name: Quintac 3399) and 5 wt % of a white pigment (titanium oxide manufactured by Dupont, trade name: Ti-Pure R103) was loaded into a B layer in the extrusion machine to extrude an elastic film (C1) having the construction of A layer/B layer/A layer=9 μm/42 μm/9 μm in total of 60 μm.

The resultant elastic film (C1) and a SIS-based hot-melt pressure-sensitive adhesive (manufactured by Bento Bantçilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (C1) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 2.

### Comparative Example 2

100 wt % of a SIS-based resin (manufactured by Zeon Corporation, trade name: Quintac 3620) was loaded into an A layer in an extrusion machine, and a formulation of 95 wt % of the SIS-based resin (manufactured by Zeon Corporation, trade name: Quintac 3620) and 5 wt % of a white pigment (titanium oxide manufactured by Dupont, trade name: Ti-Pure R103) was loaded into a B layer in the extrusion machine to extrude an elastic film (C2) having the construction of A layer/B layer/A layer=9 μm/42 μm/9 μm in total of 60 μm.

The resultant elastic film (C2) and a SIS-based hot-melt pressure-sensitive adhesive (manufactured by Bento Bantçilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (C2) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 2.

### Comparative Example 3

100 wt % of a SBS-based resin (manufactured by Kraton Polymers, Inc., trade name: Kraton D1191) was loaded into an A layer in an extrusion machine, and a formulation of 95 wt % of the SBS-based resin (manufactured by Kraton Polymers, Inc., trade name: Kraton D1191) and 5 wt % of a white pigment (titanium oxide manufactured by Dupont, trade name: Ti-Pure R103) was loaded into a B layer in the extrusion machine to extrude an elastic film (C3) having the construction of A layer/B layer/A layer=9 μm/42 μm/9 μm in total of 60 μm.

The resultant elastic film (C3) and a SIS-based hot-melt pressure-sensitive adhesive (manufactured by Bento Bantçilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (C3) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 2.

### Comparative Example 4

100 wt % of a SBS-based resin (manufactured by Kraton Polymers, Inc., trade name: Kraton 1730) was loaded into an A layer in an extrusion machine, and a formulation of 95 wt % of the SBS-based resin (manufactured by Kraton Polymers, Inc., trade name: Kraton 1730) and 5 wt % of a white pigment (titanium oxide manufactured by Dupont, trade name: Ti-Pure R103) was loaded into a B layer in the extrusion machine to extrude an elastic film (C4) having the construction of A layer/B layer/A layer=9 μm/42 μm/9 μm in total of 60 μm.

The resultant elastic film (C4) and a SIS-based hot-melt pressure-sensitive adhesive (manufactured by Bento Bantçilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (C4) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 2.

### Comparative Example 5

100 wt % of a SEBS-based resin (manufactured by Asahi Kasei Chemicals Corporation, trade name: Tuftec H1051) was loaded into an A layer in an extrusion machine, and a formulation of 95 wt % of the SEBS-based resin (manufactured by Asahi Kasei Chemicals Corporation, trade name: Tuftec H1051) and 5 wt % of a white pigment (titanium oxide manufactured by Dupont, trade name: Ti-Pure R103) was loaded into a B layer in the extrusion machine to extrude an elastic film (C5) having the construction of A layer/B layer/A layer=9 μm/42 μm/9 μm in total of 60 μm.

The resultant elastic film (C5) and a SIS-based hot-melt pressure-sensitive adhesive (manufactured by Bento Bantçilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (C5) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 2.

### Comparative Example 6

100 wt % of a SBS-based resin (manufactured by Kraton Polymers, Inc., trade name: Kraton D1191) was loaded into an A layer in an extrusion machine, and a formulation of 25 wt % of a SIS-based resin (manufactured by Zeon Corporation, trade name: Quintac 3399), 70 wt % of the SBS-based resin (manufactured by Kraton Polymers, Inc., trade name: Kraton D1191), and 5 wt % of a white pigment (titanium oxide manufactured by Dupont, trade name: Ti-Pure R103) was loaded into a B layer in the extrusion machine to extrude an elastic film (C6) having the construction of A layer/B layer/A layer=9 μm/42 μm/9 μm in total of 60 μm.

The resultant elastic film (C6) and a SIS-based hot-melt pressure-sensitive adhesive (manufactured by Bento Bantçilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (C6) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 2.

### Comparative Example 7

100 wt % of a SBS-based resin (manufactured by Kraton Polymers, Inc., trade name: Kraton D1191) was loaded into an A layer in an extrusion machine, and a formulation of 50 wt % of a SIS-based resin (manufactured by Zeon Corporation, trade name: Quintac 3399), 45 wt % of the SBS-based resin (manufactured by Kraton Polymers, Inc., trade name: Kraton D1191), and 5 wt % of a white pigment (titanium oxide manufactured by Dupont, trade name: Ti-Pure R103) was loaded into a B layer in the extrusion machine to extrude an elastic film (C7) having the construction of A layer/B layer/A layer=9 μm/42 μm/9 μm in total of 60 μm.

The resultant elastic film (C7) and a SIS-based hot-melt pressure-sensitive adhesive (manufactured by Bento Bantçilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (C7) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 2.

### Comparative Example 8

100 wt % of a SBS-based resin (manufactured by Kraton Polymers, Inc., trade name: Kraton D1191) was loaded into an A layer in an extrusion machine, and a formulation of 65 wt % of an olefin-based resin (manufactured by Exxon Mobil Corporation, trade name: Vistamaxx 6202), 30 wt % of a SBS-based resin (manufactured by Kraton Polymers, Inc., trade name: Kraton 1730), and 5 wt % of a white pigment (titanium oxide manufactured by Dupont, trade name: Ti-Pure R103) was loaded into a B layer in the extrusion machine to extrude an elastic film (C8) having the construction of A layer/B layer/A layer=9 μm/42 μm/9 μm in total of 60 μm.

The resultant elastic film (C8) and a SIS-based hot-melt pressure-sensitive adhesive (manufactured by Bento Bantgilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (C8) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 2.

### Comparative Example 9

100 wt % of an olefin-based resin (manufactured by Japan Polypropylene Corporation, trade name: Wintec WFX4) was loaded into an A layer in an extrusion machine, and a formulation of 95 wt % of a SBS-based resin (manufactured by Kraton Polymers, Inc., trade name: Kraton D1191) and 5 wt % of a white pigment (titanium oxide manufactured by Dupont, trade name: Ti-Pure R103) was loaded into a B layer in the extrusion machine to extrude an elastic film (C9) having the construction of A layer/B layer/A layer=9 μm/42 μm/9 μm in total of 60 μm.

The resultant elastic film (C9) and a SIS-based hot-melt pressure-sensitive adhesive (manufactured by Bento Bantçilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (C9) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 2.

### Comparative Example 10

100 wt % of an olefin-based resin (manufactured by Japan Polyethylene Corporation, trade name: Novatec LD LC720) was loaded into an A layer in an extrusion machine, and a formulation of 95 wt % of a SBS-based resin (manufactured by Kraton Polymers, Inc., trade name: Kraton D1191) and 5 wt % of a white pigment (titanium oxide manufactured by Dupont, trade name: Ti-Pure R103) was loaded into a B layer in the extrusion machine to extrude an elastic film (C10) having the construction of A layer/B layer/A layer=9 μm/42 μm/9 μm in total of 60 μm.

The resultant elastic film (C10) and a SIS-based hot-melt pressure-sensitive adhesive (manufactured by Bento Bantgilik, trade name: AC280) were subjected to the above-mentioned <Forming Conditions>, and a portion (A) in which the hot-melt pressure-sensitive adhesive had been applied onto the whole surface (7 g/m²) with a width of 30 mm was cut at the middle to provide a stretchable laminate (C10) having two portions (A1) and (A2), in which the hot-melt pressure-sensitive adhesive had been applied onto the whole surface (7 g/m²) with a width of 15 mm from both ends, and a portion (B), in which the hot-melt pressure-sensitive adhesive had been applied (15 g/m²) between the (A1) and the (A2) with a width of 41 mm in a striped manner (pressure-sensitive adhesive width: 1 mm, interval: 1 mm), on both surfaces of the elastic film.

The results are shown in Table 2.

TABLE 1

| | | Example | | | | | | | | | |
|---|---|---|---|---|---|---|---|---|---|---|---|
| | | 1 | 2 | 3 | 4 | 5 | 6 | 7 | 8 | 9 | 10 |
| A layer resin | | Vistamaxx | ← | ← | ← | Vistamaxx | Vistamaxx | Vistamaxx | Vistamaxx | Vistamaxx | Vistamaxx |
| (1) | | 6202 | | | | 7010 | 7010 | 7010 | 7010 | 7010 | 6202 |
| A layer resin | | — | — | Tafmer | ← | — | — | — | — | — | — |
| (2) | | | | PN-3560 | | | | | | | |
| B layer resin | | Vistamaxx | ← | Vistamaxx | ← | ← | ← | Quintac | Quintac | — | Vistamaxx |
| (1) | | 6202 | | 3000 | | | | 3399 | 3399 | | 6202 |
| B layer resin | | Tafmer | ← | ← | ← | ← | ← | Kraton | Kraton | Kraton | Kraton |
| (2) | | PN-3560 | | | | | | D1191 | D1191 | D1191 | 1730 |
| B layer resin | | TiO2 | ← | ← | ← | ← | ← | ← | ← | ← | ← |
| (3) | | | | | | | | | | | |
| A layer | | 100/0 | 100/0 | 50/50 | 50/50 | 100/0 | 100/0 | 100/0 | 100/0 | 100/0 | 100/0 |
| formulation | | | | | | | | | | | |
| (1)/(2) | | | | | | | | | | | |
| B layer | | 65/30/5 | ← | 45/50/5 | 25/70/5 | 45/50/5 | 25/70/5 | 50/45/5 | 50/45/5 | 0/95/5 | 65/30/5 |
| formulation | | | | | | | | | | | |
| (1)/(2)/(3) | | | | | | | | | | | |
| A/B/A thickness | μm | 9/42/9 | 6.75/31.5/ | 6.75/31.5/ | 6.75/31.5/ | 6.75/31.5/ | 6.75/31.5/ | 6.75/31.5/ | 9/42/9 | 6.75/31.5/ | 6.75/31.5/ |
| | | | 6.75 | 6.75 | 6.75 | 6.75 | 6.75 | 6.75 | | 6.75 | 6.75 |
| Total thickness | μm | 60 | 45 | 45 | 45 | 45 | 45 | 45 | 60 | 45 | 45 |
| of elastic film | | | | | | | | | | | |
| Kind of hot-melt | | AC280 | AC280 | AC280 | AC280 | AC280 | AC280 | AC280 | AC280 | AC280 | AC280 |
| pressure-sensi- | | | | | | | | | | | |
| tive adhesive | | | | | | | | | | | |
| Type of | | Carded 24 | Carded 24 | Carded 24 | Carded 24 | Carded 24 | Carded 24 | Carded 24 | Carded 24 | Carded 24 | Carded 24 |
| non-woven fabric | | g/m² | g/m² | g/m² | g/m² | g/m² | g/m² | g/m² | g/m² | g/m² | g/m² |
| Dropping oil | | ∘ | ∘ | ∘ | ∘ | ∘ | ∘ | ∘ | ∘ | ∘ | ∘ |
| resistance | | | | | | | | | | | |
| After 10 min | | | | | | | | | | | |
| 40° C. holding | min | >180 | >180 | >180 | >180 | >180 | >180 | >180 | >180 | >180 | >180 |
| force test | | | | | | | | | | | |
| Retention time | | | | | | | | | | | |
| 40° C. holding | | ∘ | ∘ | ∘ | ∘ | ∘ | ∘ | ∘ | ∘ | ∘ | ∘ |
| force test | | | | | | | | | | | |
| Presence or | | | | | | | | | | | |
| absence of film | | | | | | | | | | | |
| break | | | | | | | | | | | |
| Elasticity | | ∘ | ∘ | ∘ | ∘ | ∘ | ∘ | ∘ | ∘ | ∘ | ∘ |

TABLE 2

| | | Comparative Example | | | | | | | | | |
|---|---|---|---|---|---|---|---|---|---|---|---|
| | | 1 | 2 | 3 | 4 | 5 | 6 | 7 | 8 | 9 | 10 |
| A layer resin | | Quintac | Quintac | Kraton | Kraton | Tuftec | Kraton | Kraton | Kraton | Wintec | Novatec LD |
| | | 3399 | 3620 | D1191 | 1730 | H1051 | D1191 | D1191 | D1191 | WFX4 | LC720 |
| B layer resin | | Quintac | Quintac | Kraton | Kraton | Tuftec | Quintac | Quintac | Vistamaxx | Kraton | Kraton |
| (1) | | 3399 | 3620 | D1191 | 1730 | H1051 | 3399 | 3399 | 6202 | D1191 | D1191 |
| B layer resin | | — | — | — | — | — | Kraton | Kraton | Kraton | — | — |
| (2) | | | | | | | D1191 | D1191 | 1730 | | |
| B layer resin | | TiO2 | ← | ← | ← | ← | ← | ← | ← | ← | ← |
| (3) | | | | | | | | | | | |
| B layer | | 95/0/5 | ← | ← | ← | ← | 25/70/5 | 50/45/5 | 65/30/5 | 95/0/5 | 95/0/5 |
| formulation | | | | | | | | | | | |
| (1)/(2)/(3) | | | | | | | | | | | |
| A/B/A thickness | μm | 9/42/9 | 9/42/9 | 9/42/9 | 9/42/9 | 9/42/9 | 9/42/9 | 9/42/9 | 9/42/9 | 9/42/9 | 9/42/9 |
| Total thickness | μm | 60 | 60 | 60 | 60 | 60 | 60 | 60 | 60 | 60 | 60 |
| of elastic film | | | | | | | | | | | |
| Kind of hot-melt | | AC280 | AC280 | AC280 | AC280 | AC280 | AC280 | AC280 | AC280 | AC280 | AC280 |
| pressure-sensi- | | | | | | | | | | | |
| tive adhesive | | | | | | | | | | | |
| Type of | | Carded 24 | Carded 24 | Carded 24 | Carded 24 | Carded 24 | Carded 24 | Carded 24 | Carded 24 | Carded 24 | Carded 24 |
| non-woven fabric | | g/m² | g/m² | g/m² | g/m² | g/m² | g/m² | g/m² | g/m² | g/m² | g/m² |
| Dropping oil | | x | x | x | x | x | x | x | x | ∘ | ∘ |
| resistance | | | | | | | | | | | |
| After 10 min | | | | | | | | | | | |
| 40° C. holding | min | 65 | 69 | 30 | 37 | 55 | 21 | 45 | 94 | 180 | 180 |
| force test | | | | | | | | | | | |
| Retention time | | | | | | | | | | | |
| 40° C. holding | | x | x | x | x | x | x | x | x | ∘ | ∘ |
| force test | | | | | | | | | | | |
| Presence or | | | | | | | | | | | |
| absence of film | | | | | | | | | | | |
| break | | | | | | | | | | | |
| Elasticity | | ∘ | ∘ | ∘ | ∘ | ∘ | ∘ | ∘ | ∘ | x | x |

### INDUSTRIAL APPLICABILITY

The stretchable laminate of the present invention can be used in any appropriate article in which the effects of the present invention can be effectively utilized. That is, the article of the present invention includes the stretchable laminate of the present invention. Atypical example of such article is a sanitary article. Examples of such sanitary article include a diaper (in particular, an ear portion of a disposable diaper), a supporter, and a mask.

### REFERENCE SIGNS LIST

100 stretchable laminate
10 elastomer layer
11a outer layer
11b outer layer
12 intermediate layer
20 non-woven fabric layer
20a non-woven fabric layer
20b non-woven fabric layer
30 hot-melt pressure-sensitive adhesive

1. A stretchable laminate, including:
an elastomer layer; and
a non-woven fabric layer arranged on at least one side of the elastomer layer, wherein:
the elastomer layer comprises a plurality of layers; and
at least one outer layer of the plurality of layers contains an olefin-based elastomer.

2. The stretchable laminate according to claim 1, wherein the elastomer layer comprises three layers, and outer layers on both sides of the elastomer layer each contain the olefin-based elastomer.

3. The stretchable laminate according to claim 1, wherein a content of the olefin-based elastomer in the outer layer is from 50 wt % to 100 wt %.

4. The stretchable laminate according to claim 3, wherein the content of the olefin-based elastomer in the outer layer is from 80 wt % to 100 wt %.

5. The stretchable laminate according to claim 4, wherein the content of the olefin-based elastomer in the outer layer is from 95 wt % to 100 wt %.

6. The stretchable laminate according to claim 1, wherein the olefin-based elastomer comprises an α-olefin-based elastomer.

7. The stretchable laminate according to claim 6, wherein the α-olefin-based elastomer comprises at least one kind selected from an ethylene-based elastomer, a propylene-based elastomer, and a 1-butene-based elastomer.

8. The stretchable laminate according to claim 7, wherein the α-olefin-based elastomer comprises a propylene-based elastomer.

9. The stretchable laminate according to claim 6, wherein the α-olefin-based elastomer is produced by using a metallocene catalyst.

10. The stretchable laminate according to claim 1, wherein an elastomer layer except the outer layer containing the olefin-based elastomer in the elastomer layer contains 50 wt % to 100 wt % of an elastomer resin.

11. The stretchable laminate according to claim 10, wherein the elastomer layer except the outer layer containing the olefin-based elastomer in the elastomer layer contains 80 wt % to 100 wt % of an elastomer resin.

12. The stretchable laminate according to claim 11, wherein the elastomer layer except the outer layer containing the olefin-based elastomer in the elastomer layer contains 95 wt % to 100 wt % of an elastomer resin.

13. The stretchable laminate according to claim 1, wherein an elastomer layer except the outer layer containing the olefin-based elastomer in the elastomer layer contains an elastomer resin, and the elastomer resin comprises at least one kind selected from an olefin-based elastomer, a styrene-based elastomer, a vinyl chloride-based elastomer, a urethane-based elastomer, an ester-based elastomer, and an amide-based elastomer.

14. The stretchable laminate according to claim 13, wherein the elastomer resin comprises at least one kind selected from the olefin-based elastomer and the styrene-based elastomer.

15. The stretchable laminate according to claim 1, wherein the elastomer layer has a thickness of from 20 μm to 200 μm.

16. The stretchable laminate according to claim 15, wherein the elastomer layer has a thickness of from 30 μm to 100 μm.

17. The stretchable laminate according to claim 1, wherein the non-woven fabric layer is formed of a non-woven fabric having a basis weight of from 10 gsm to 30 gsm.

18. The stretchable laminate according to claim 1, further comprising a hot-melt pressure-sensitive adhesive between the elastomer layer and the non-woven fabric layer.

19. A stretchable laminate, including:
an elastomer layer; and
a non-woven fabric layer arranged on at least one side of the elastomer layer, wherein when the stretchable laminate is bonded and fixed onto a glass plate in a state of being extended by 100%, and 0.5 mL of a baby oil (manufactured by Pigeon Corporation, Baby Oil P, main component: caprylic/capric triglyceride) is dropped onto a surface of the stretchable laminate, the stretchable laminate is free from rupturing 10 minutes after the dropping.

20. An article, comprising the stretchable laminate of claim 1.
